(19) United States
(12) Patent Application Publication
(10) Pub. No.: US 2018/0228371 A1
(43) Pub. Date: Aug. 16, 2018

(54) IMPLANTABLE DEVICE

(71) Applicant: Micell Technologies, Inc., Durham, NC (US)

(72) Inventor: James B. McClain, Ocracoke, NC (US)

(73) Assignee: Micell Technologies, Inc., Durham, NC (US)

(21) Appl. No.: 15/750,096

(22) PCT Filed: Aug. 3, 2016
(86) PCT No.: PCT/US2016/045292

§ 371 Date: Feb. 2, 2018

Related U.S. Application Data
(60) Provisional application No. 62/201,813, filed on Aug. 6, 2015.

(51) Int. Cl. A61B 5/00; A61B 5/0215; A61B 5/024

(52) U.S. Cl. CPC A61B 5/0028; A61B 5/0215; A61B 5/6862; A61B 2560/0214; A61B 5/02444; A61B 5/686; A61B 5/4839; A61B 5/0022

(57) ABSTRACT

An implantable device including a sensing element, transmitter and power element is disclosed. The device may allow for monitoring of one or more body conditions in a minimally invasive manner, while a receiver may receive such information. Methods of utilizing device are also disclosed.

### CROSS-REFERENCE OF RELATED APPLICATION

The present application claims the benefit of the filing date of U.S. Provisional Application No. 62/201,813, filed Aug. 6, 2015, entitled IMPLANTABLE DEVICE, the disclosure of which is hereby incorporated herein by reference.

### BACKGROUND OF THE INVENTION

The present invention relates generally to implantable medical devices that provide a sensing/telemetry function. In particular, the present invention is related to cardiovascular implants, such as a stent, cardiac support mesh or pericardial implant that can provide real time information pertaining to signals that monitor cardiovascular health (flow, rhythm, arterial patency, etc. . . . ).

Stents are a commonly employed device for treating cardiovascular disease. Typically, a stent is placed within a coronary artery to keep open the arterial passage in order to allow for unimpeded blood flow to the heart. Many different types of stents have been developed over the years, including expandable, self-expanding and absorbable stents and stents including pharmacologically or biologically beneficial coatings. Some examples of these stents are disclosed in U.S. Pat. Nos. 8,298,565; 8,758,429; 8,834,913; 8,257,424; and 8,852,625, U.S. Patent Application Publication Nos. 2009/0292351; 2010/0256748; 2010/0272778; 2011/0257732; 2012/0323311; 2010/0063580; 2010/0211164; 2010/0298928; 2015/0030757; 2011/0190864; 2011/0264190; 2014/0257465; 2014/0277401; 2014/0343667; and 2015/0087671, and U.S. patent application Ser. No. 14/402,832. The disclosures of the foregoing are hereby incorporated by reference herein.

Although stents remain an important device for treating cardiovascular disease, current stents are limited to physical and/or pharmacological modes of action; they are not intelligent devices. Put another way, while serving to physically reestablish or maintain blood flow through a blood vessel, stents generally do not do more. However, monitoring the operation of a patient's cardiovascular system, especially after the implantation of a stent, is of the utmost importance. Parameters like blood flow, blood pressure, heart rhythm and operation, arterial patency, and the like can determine whether the stent is providing the benefit it is designed to, as well as to determine the overall health of the patient.

It would be beneficial to provide a “smart” stent that could, inter alia, monitor the operation of a patient's cardiovascular system.

### BRIEF SUMMARY OF THE INVENTION

The present invention discloses “smart” implantable medical devices. In particular, several implantable stents are disclosed that include the ability to monitor bodily functions/levels. While specifically discussed in the context of stents, other implants are contemplated. The use of such devices may allow for active monitoring after implantation from an otherwise passive device. Other medical implants contemplated include cardiac meshes, particularly designed for the measurement of inflammation and rhythm. Because of their proximity to the heart, these meshes are preferably designed to be powered by the motion of the heart. For instance, like other implants according to the present invention, such meshes may include a power source that is charged (e.g., wind-up, piezoelectric, etc. . . . ) by the beating of the heart.

A first aspect of the present invention is an implantable medical device including a body, a sensing element for monitoring a bodily function, a transmitter and a power element. In certain embodiments according to the first aspect, the power element may convert energy derived from a body of a patient. The power element may include a battery that is charged from external to a body of a patient or it may be charged from external to a body of a patient. The sensing element may be formed integral with the body. In certain embodiments, the device may be a stent and the body may be a tubular body. The sensing element may monitor blood flow (arterial patency) via monitoring pressure exerted by the flowing blood on through, for instance a stented artery, whether there is a blockage formed through the tubular body or heart rhythm. Still other embodiments may further include a receiver in communication with the transmitter. The receiver may be a dedicated monitor or a handheld device, such as a smart phone. The device may also be a cardiac mesh that includes a sensing element designed to monitor heart rhythm or the like.

### BRIEF DESCRIPTION OF THE SEVERAL VIEWS OF THE DRAWINGS

The foregoing summary, as well as the following detailed description of the preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

Referring to the figures, wherein like reference numerals represent like parts throughout the several views:

FIG. 1 is a block diagram illustrating one embodiment of the present invention.

FIG. 2 is a block diagram illustrating the use of a device according to the present invention.

### DETAILED DESCRIPTION OF THE INVENTION

Reference will now be made in detail to the preferred embodiments of the present invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import.

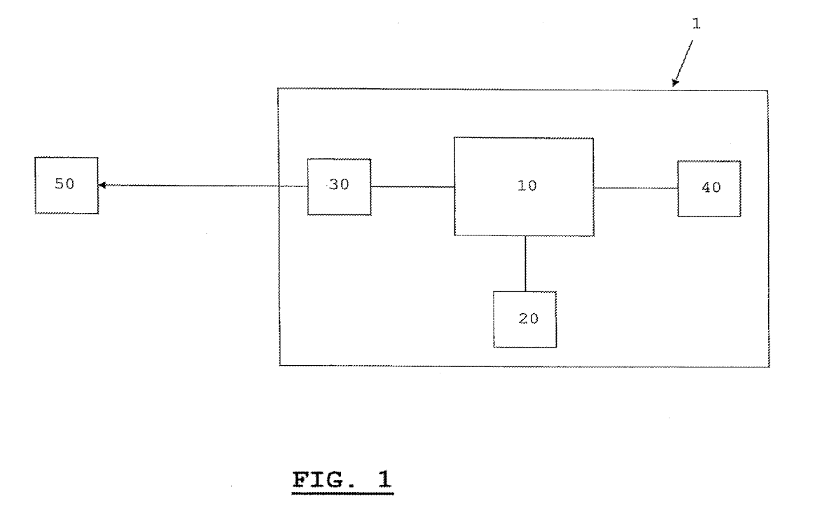

Several different implantable device examples according to the present invention will be disclosed herein. In general, with reference to FIG. 1, an implant (e.g., a stent) 10 according to the present invention includes a sensing element 20, a transmitter 30 and a power element 40. Transmitter 30 is designed to transmit information collected by sensing element 20 so that such can be received by a receiver 50. In certain embodiments, sensing element 20 and transmitter 30 may be different elements coupled together via an electrical connection or the like, or the two components may be part of the same set of circuitry. Power element 40 may likewise be part of the same set of circuitry or a separate component electrically connected with sensing element 20 and transmitter 30. Power element 40 is designed to provide the necessary electrical power to sensing element 20 and transmitter 30. In one embodiment, implant 10 is a stent and is designed for implantation within the cardiovascular system of a patient 1. Information is collected from within the cardiovascular system by sensing element 20 and is transmitted to receiver 50 by transmitter 30. This information may pertain to, inter alia, blood flow, blood pressure, the content of the blood, the patency of the artery, or the like.

Although largely discussed below in the context of stent structures, the present invention has applicability to many different types of implantable medical devices. For instances, certain embodiments may be designed to serve the same function as existing cardiac meshes. Such devices may in fact be better designed to monitor operation of the heart (e.g., rhythm) because of their location when implanted. Likewise, it is envisioned to design both surgical and percutaneous valves in accordance with the present invention. Such devices may be configured to sense inflammation and/or flow parameters such as leakage and ejection fraction. Furthermore it is entirely within the purview of the present invention to provide a standalone sensor that is not otherwise coupled to a device. This type of device could be implanted percutaneously, surgically, laproscopically, either alone or in conjunction with another medical intervention. Of course, it is contemplated to provide such a sensor with a power element and transmitter to allow for the sensing and transmitting operations noted above.

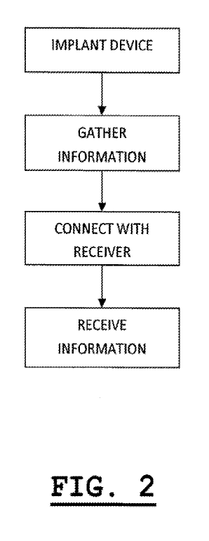

A method of utilizing such a device is generally depicted in FIG. 2. There, a device 10 is implanted in a patient, information pertaining to the patient is obtained by sensing element 20, connection between transmitter 30 and receiver 50 is established and the collected information is received by receiver 50. It is noted that the sensing connecting and receiving steps may be performed at different intervals or at a specific desired time. As such, the patient, a doctor or other medical professional may gather information continuously or at a specific desired time. For instance, device 10 may be designed to only collect and transmit information when wirelessly connected with a receiver 50 during a predefined set of medical conditions (e.g., atrial fibrillation, loss of arterial patency, etc.) or a patient visit. Alternatively, device 10 may be configured to be in consistent contact with a receiver, such a smart phone or the like, so that data collection can occur over a longer period of time. Of course, any connections with an external receiver should be done securely so as to prevent any unwanted access of the device.

Several different examples according to the present invention will now be discussed. These are merely exemplary of the present invention, and other variations of devices 10 may still fall within the scope hereof. Likewise, while specific purposes for each example are outlined, it is to be understood that different devices may be directed to different purposes, and certain may in fact serve multiple purposes. Devices according to the present invention are not limited to just those examples provided below.

### Example 1

In one particular embodiment according to the present invention, a device is provided that is for collecting and transmitting information pertaining to heart rhythm. The device may be in the form of a stent, which is to be implanted in a coronary artery or vein. The sensing element included on the stent may collect the heart rhythm information in various ways. For instance, physical motions (e.g., pulsation rate, rhythm, patterns) or electronic or electrochemical signals (e.g., EKG) may be detected. In the case of the former, the sensing element may be placed on the stent so that it directionally aligned to the arterial lumen. In the case of the latter, the sensing element may include a series of contacts that allow for an operation similar to that of existing EKG monitors. Of course, other types of sensing elements may also be employed.

According to this embodiment, the stent may also include a transmitter in communication with the sensing element. The transmitter is preferably capable of communicating with many different types of receivers via well-known communication technology, for instance, Wi-Fi, Bluetooth, RF, IR or the like. Likewise, a receiver equipped with the same communication technology could take many forms, yet still communicate with the transmitter. By way of example, the receiver may be a dedicated receiver for use only with the devices and its transmitter, or may be a more general multipurpose device such a smart phone equipped with a receiver app. For devices where a constant monitoring of heart rhythm is desired, a wearable or otherwise easily transportable (e.g., a belt worn device or smart phone) receiver are preferable. Other receivers according to the present invention may only receiver information (or force the sensing element to collect information) upon an input from the patient or medical professional. Such input could be provided in the context of a physician visit or via remote communication with the implantable device system. This may be desirable for use during in patient visits with a doctor or other medical professional. Of course, any device/receiver combination should be connected via an encrypted connection so as to avoid the unwanted access of the device.

A power element is provided with a device according to this example in order to provide electrical power to the sensing element and transmitter. In one embodiment, the power element may be in the form of a battery, which may have a finite power supply life or may be rechargeable. With respect to the latter, it is envisioned to employ a wireless charging module to recharge the battery. For devices where only in-patient sensing/transmitting is desired, an entirely wireless power element may be employed. Of course, other power elements are also contemplated, such as piezoelectric conversion elements that can take mechanical motion of the body (e.g., heartbeats, motion by the patient etc. . . . ) and convert it to electrical energy. For instance, a device according to the present invention may be implanted in an area of the body where motion is common (e.g., legs, arms, etc. . . . ) and motion (e.g., bending, swinging, etc. . . . ) can provide stress to a power element that converts such stress to usable energy. Similarly, an electrochemical device that harvests the electronic signals pervading the heart's muscle and nerve tissues can be employed. The device may also employ a power source akin to a self-winding watch (i.e., a kinetic wind up gear). In such a device, the kinetic drive can be powered from the heartbeats or body motions of the patient. Alternatively, an externally-coupled charging system could be employed. One example of the later is a strap that can be provided for “winding up” the source, which essentially includes a magnet that oscillates or orbits external to the body to wind the device. Finally, it is also envisioned to provide a recharging port that is accessible at or near the skin level of the patient to allow for charging of the battery with a direct electrical connection or with near-field wireless power. Of course, certain stent applications might make such a construct difficult given the placement of a stent in a body lumen. However, other embodiment implants according to the present invention may more easily employ such a design.

While the sensing element, transmitter and power element are discussed above as being integral with the implantable device, it is noted that either or both elements may be located away therefrom. For instance, it is contemplated to locate the power element and transmitter closer to the skin level so as to aid in charging and transmission, respectively. Likewise, the sensing element(s) may be located on or near the device itself. This is particularly useful where something other than directly within the vicinity needs to be monitored (e.g., EKG information from a stent implanted within a coronary artery). Again, such a design may be more readily employed in devices other than stents.

Although this first example is largely discussed in the context of a stent implanted within a coronary artery, it is to be understood that the device itself can be many different types of implantable devices. For instance, instead of being a stent implanted in a coronary artery, the device may be a stent implanted within a vein, which may in fact allow for better monitoring of cardiac rhythm in cases where a medical intervention in the coronary arteries is not clinically indicated. Likewise, the device could be a surgical implant on the pericardium, a device implanted within a chamber of the heart, or a cardiac support mesh. Additionally, as is alluded to above, the device may be designed so as to continuously or periodically monitor heart rhythm (e.g., ever 15 seconds), and continuously or periodically transmit data to a receiver. For periodically transmitted operations, the device may be provided with a memory for maintaining a record of readings taken, but not transmitted. Devices according to this example may allow patient monitoring of rhythm-based clinical indications, such as assorted fibrillation disorders and/or events. In fact, it is envisioned that the device may only transmit if a certain set of signals is sensed. For instance, a blockage may cause the heart rhythm to fluctuate in a manner that would be transmitted to the receiver. The receiver may be provided with a system that initiates prescription or delivery of a pharmaceutical (e.g., a calcium channel blocker, a beta blocker or digoxin) and/or a medical response (e.g., medical or electrical cardioversion in response to the data received. While it is possible for the device itself to allow for a pharmaceutical to be released or medical response to be provided, other embodiments simply provide an indication to the patient or medical professionals that may be monitoring the data that such treatments should be sought. Thereafter, a medical professional may administer such accordingly.

Stents according to the present invention may be any type of known stent construction and may include coatings like those described in the above-incorporated references. The stents may also be utilized in conjunction with a balloon for expansion of the stent in situ. Likewise, for other types of devices, it is to be understood that all known variations thereof may be employed in connection with the present invention.

### Example 2

In another embodiment according to the present invention, a device is provided that is for collecting and transmitting detecting blood flow. This particular embodiment may be particularly relevant for determining blockages and/or arterial patency information for arteries (e.g., advance restenosis and/or acute thrombosis in a stented artery). Of course, although specifically discussed in connection with arteries, this embodiment may be useful for determining flow in other body lumens (e.g., veins (for DVT), urinary tract, biliary tract, digestive tract, reproductive system, etc. . . . ).

The construction and operation of this second embodiment may be substantially similar to the above-described first embodiment, with the exception that the sensing elements are preferably designed to monitor blood flow via measurement of pressure(s). For instance, it is contemplated to provide pressure sensors on both ends of the stent. This would allow for a blockage within the stent to be identified by reviewing a pressure drop across the stent. Additionally, a blockage can be identified by determining a lower difference between diastolic and systolic blood pressure at the proximal or distal end of the stent.

Moreover, it is contemplated to correlate the cross-sectional density of biological material within the lumen of the stent to confirm the absence or amount of tissue/clot material across the diameter of the stent (tissue build up from restenosis and/or clot of thrombosis would be denser than blood). This can be achieved by utilizing optical sensors diametrically opposed across the diameter of the stent or sensors to monitor other energies that would be affected by the different density of tissues versus blood (sound, ultrasound, etc. . . . ).

As noted above, this second embodiment otherwise operates similar to the first embodiment described above. For instance, should a detrimental condition be identified, the receiver may be provided with a system that initiates prescription of or provides a pharmaceutical (e.g., a thrombolytic drug for thrombosis—Eminase (anistreplase), Retavase (reteplase), Streptase (streptokinase, kabikinase), t-PA (class of drugs that includes Activase), TNKase (tenecteplase), Abbokinase, Kinlytic (rokinase)) and/or medical response (e.g., percutaneous intervention, bypass surgery). While it is possible for the device itself to allow for a pharmaceutical to be released or medical response to be provided, other embodiments simply provide an indication to the patient or to medical professionals that such treatments should be sought. Thereafter, a medical professional may administer such accordingly.

### Example 3

In another embodiment according to the present invention, a device is provided that is for collecting and transmitting information pertaining to tissue in-growth, plaque formation, vulnerable plaque composition and/or neoatheroma progression in or around a stented artery. Again, while discussed specifically in the context of an artery, this embodiment may be useful for determining flow in other body lumens (e.g., veins (for DVT), urinary tract, biliary tract, digestive system, reproductive system, etc. . . . ).

In this embodiment, the sensing element(s) are designed to assess and measure the tissues in and around an implanted stent. In particular, when measuring the formation of plaque, the extent, density, calcification, lipid make-up (e.g., neoatheroma, vulnerable plaque), integrity of fibrin cap (e.g., in the case of vulnerable plaque) may be measured/monitored. The same type of optical or sound/ultrasound utilized in the second example discussed above can be utilized here in connection with this third example. In addition, the sensing elements may be capable of Raman spectroscopy (or more simply IR spectroscopy) to more directly characterize the composition of local tissues.

As noted above, this third embodiment otherwise operates similar to the first and second embodiments described above. For instance, should a detrimental condition be identified, the receiver may be provided with a system that initiates prescription or provides a pharmaceutical treatment (e.g., aggressive statin therapies, anti-platelet regimen, etc. . . . ) and/or medical response (e.g., percutaneous intervention, bypass surgery). While it is possible for the device itself to allow for a pharmaceutical to be released or medical response to be provided, other embodiments simply provide an indication to the patient that such treatments should be sought. Thereafter, a medical professional may administer such accordingly.

### Example 4

In another embodiment according to the present invention, a device is provided that is for collecting and transmitting information pertaining local inflammation or atherosclerosis, neoatheroma or vulnerable plaque in tissue in/near stented arteries. Again, while discussed specifically in the context of a coronary artery, this embodiment may be useful for determining flow in other body lumens (e.g., peripheral arteries, renal arteries, carotid arteries, veins (for DVT), urinary tract, biliary tract, digestive system, reproductive system, etc. . . . ).

The sensing element(s) according to this embodiment may assess the foregoing by monitoring the temperature, pH, pH heterogenity of the local tissue and/or composition and/or biomarkers within such tissue (e.g., C-reactive protein). This fourth embodiment otherwise operates similar to the other embodiments described above. For instance, should a detrimental condition be identified, the receiver may be provided with a system that initiates prescription of, or provides a pharmaceutical (e.g., an anti-inflammatory) and/or medical response (e.g., percutaneous intervention, bypass surgery). While it is possible for the device itself to allow for a pharmaceutical to be released or medical response to be provided, other embodiments simply provide an indication to the patient that such treatments should be sought. Thereafter, a medical professional may administer such accordingly.

As noted above, devices according to the present invention may encompass many different forms. While discussed largely in the context of stents, other types of devices are clearly contemplated, such as implants, meshes or the like. Moreover, utilization of the devices disclosed herein can include the performance of different steps. For instance, collection of data by a sensing element may occur continuously or upon specific request of the element. Receivers according to the present invention may also take on many different forms, including smart phones or other handheld devices.

It is also to be understood that the disclosure set forth herein includes all possible combinations of the particular features described. For example, where a particular feature is disclosed in the context of a particular aspect, arrangement, configuration, or embodiment, or a particular claim, that feature can also be used, to the extent possible, in combination with and/or in the context of other particular aspects, arrangements, configurations, and embodiments of the invention, and in the invention generally.

Furthermore, although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1.-15. (canceled)

16. An implantable medical device comprising:
a body having a coating including a polymer and a macrolide immunosuppressive drug dispersed within the polymer;
a sensing element for monitoring a bodily function;
a transmitter; and
a power element.

17. The implantable medical device according to claim 16, wherein the power element converts energy derived from a body of a patient.

18. The implantable medical device according to claim 16, wherein the power element includes a battery.

19. The implantable medical device according to claim 18, wherein the battery is charged from external to a body of a patient.

20. The implantable medical device according to claim 18, wherein the battery is charged from energy derived from a body of a patient.

21. The implantable medical device according to claim 16, wherein the sensing element is formed integral with the body.

22. The implantable medical device according to claim 16, wherein the sensing element monitors blood pressure.

23. The implantable medical device according to claim 16, wherein the device is a stent and the body is a tubular body.

24. The implantable medical device according to claim 23, wherein the sensing element determines whether there is a blockage formed through the tubular body.

25. The implantable medical device according to claim 16, wherein the sensing element monitors heart rhythm.

26. The implantable medical device according to claim 16, further comprising a receiver in communication with the transmitter.

27. The implantable medical device according to claim 26, wherein the receiver is a handheld device.

28. The implantable medical device according to claim 27, wherein the receiver is a smart phone.

29. The implantable medical device according to claim 16, wherein the device is a cardiac mesh.

30. The implantable medical device according to claim 29, wherein the sensing element monitors heart rhythm.
